(19) United States
(12) Patent Application Publication
(10) Pub. No.: US 2024/0102895 A1
(43) Pub. Date: Mar. 28, 2024

(54) SWAB COLLECTION MEDIA FOR CAPTURE OF AIRBORNE PARTICLE SAMPLES

(71) Applicant: Aerosol Devices Inc., Fort Collins, CO (US)

(72) Inventors: Dominick Dalton Reyes HESKETT, Greeley, CO (US); Patricia Bea KEADY, Fort Collins, CO (US)

(21) Appl. No.: 18/275,713

(22) PCT Filed: Feb. 2, 2022
(86) PCT No.: PCT/US2022/014976

§ 371 Date: Aug. 3, 2023

Related U.S. Application Data
(60) Provisional application No. 63/145,406, filed on Feb. 3, 2021.

(51) Int. Cl. G01N 1/22

(52) U.S. Cl. CPC G01N 1/2214; G01N 1/2208; G01N 2001/2282; G01N 2001/2285

(57) ABSTRACT

The present invention features a device and method for directly collecting airborne particles onto a swab collection substrate. Prior to collection, water vapor may be condensed onto the particles to increase their average diameter. The particles are expelled from one or more acceleration nozzles for gentle impaction onto the swab collection substrate and may be further analyzed through chemical or biological assays.

### CROSS-REFERENCE TO RELATED APPLICATION

This application claims the benefit of priority to U.S. Provisional Patent Application No. 63/145,406 filed on Feb. 3, 2021, the entirety of which is incorporated herein by reference.

### STATEMENT OF GOVERNMENTAL SUPPORT

This invention was made with government support under SBIR grant numbers IIP 1853240 and IIP 2027696 awarded by The National Science Foundation. The government has certain rights in the invention.

### FIELD

The present disclosure relates to detection of airborne particles, and more particularly, to direct sampling of aerosolized viruses, bacteria, fungal spores, toxins, metabolites, and other biological particles onto a swab collection substrate for subsequent genomic analysis, culturing, and other biological assays.

### BACKGROUND

Airborne particles are ubiquitous indoors and outdoors. Pathogens and pollutants can be carried by airborne particles and remain suspended in the air for a long time. It is important to monitor the level of these pathogens and pollutants in the air. Currently, the concentrations and content of airborne particles can be measured and assessed by collecting them into culture media by samplers (e.g., impactors). Airborne particles that settle on a surface or collect on a filter media can be swiped with a swab to collect a qualitative sample of the particles (Yamayoshi et al 1984). A typical analysis would be for the determination of the presence of a specific pathogen, or to characterize the relative microbial diversity within the sample, both using molecular analysis. A surface swab swipe is incapable of providing a quantitative sample that can be used to determine viral or microbial load from a fixed volume of air. Jansson 2020 discusses the impact of swab material on surface sampling whereby the recovery data is contradictory and highly dependent on the swab material, surface characteristics and the bacteria or virus analyzed. Examples of surface swab sampling of aerosol particles include: WHO guidance on sampling for SARV-CoV-2 virus on surfaces by health care professionals (WHO 2020), and a MetaSUB citizen science initiative to sample and map the urban metagenome as part of a global study on the genetic dynamics of the world's cities (www.metasub.org).

Airborne particles can be subject to an inertial impactor for particle collection before they can be subject to further analysis. An excellent description of an inertial impactor for particle collection is given in the excerpt below, taken directly from Aerosol Measurement (Marple and Olson, 2011, edited by Kulkarni, Baron and Willeke, p 134). A history of the first 100 years of inertial impactors is given by Marple 2004.

Inertial impaction is an effective method of collecting biological particles larger than the cut size of the impactor, including most bacteria, fungal spores, allergens, and fomites or droplets that may contain biological particles. Inertial impaction is more challenging for collecting particles that are smaller than the impactor collection cut size. This may include small bacteria, viruses, proteins, metabolites, toxins and fragments of bio-particles. Bioaerosol impactors are available from a variety of vendors and several are listed in the NIOSH Manual of Analytical Methods (Jensen and Schafer 1998). The commercial bioaerosol impaction samplers typically cannot collect particles smaller than approximately 1 micrometer diameter.

If ultrafine particles can be enlarged to a physical size larger than the impactor cut size, the collection efficiency can be enhanced to near 100%. This can be accomplished by condensing a supersaturated vapor onto the particle while airborne to form a larger droplet. Condensation particle growth is a principle that has been used to enlarge ultrafine atmospheric particles for optical sensing (Aitken 1888, McMurry 2000), and more recently has been applied to particle collection (Eiguren-Fernandez et al 2015). The condensing fluid may be an organic (e.g. alcohols, ethylene glycol) or inorganic (e.g. water).

The most common methods of creating the conditions of supersaturation and condensation in a gas are adiabatic expansion, conductive cooling, thermal diffusion, and mixing of a vapor saturated hot and cold gas stream (Cheng 2011). Modern condensation particle counters use condensation growth coupled with direct optical particle sensing for measuring ultrafine particle concentration in the atmosphere. Applications include air quality and climate research, indoor air quality investigations, filter efficiency testing, clean room monitoring, and basic aerosol research. Condensation of water vapor onto particles in situ has been employed in mixing-type systems (Weber et al 2001, Romay et al 2016) and thermal diffusion systems (Hering and Stolzenburg 2005, Ahn 2009, Lewis and Hering 2013, Hering et al 2014).

Laminar-flow, water condensation particle growth capture is a method that collects a concentrated sample of aerosol particles with high collection efficiency over a wide size range from 5 to 10,000 nanometers in diameter (Eiguren-Fernandez et al 2014a). The collection substrate can be liquid or a solid. The particle sample is subsequently analyzed for physical, chemical or biological characteristics (Eiguren-Fernandez 2014a and 2014b, Hecoban et al 2016, Van Schooneveld et al 2018, Zheng et al 2018, Pan et al 2018).

Droplets encapsulating a particle that were created in a condensation growth system flow through an accelerating nozzle and are gently impacted onto a swab providing a concentrated collection of the particles on the absorbent swab tip. The swab tip absorbs the droplet fluid leaving solid particles on the outer surface of the swab. The gentle impaction and droplet absorption prevents particle bounce common with classical impaction of solid particles on dry solid surfaces. The gentle impaction of the droplet onto the swab also prevents mechanical stress to biological particles and keeps the cells intact. Soluble particles that are dissolved in the droplet are absorbed into the swab material. Analysis of the airborne particle sample that is collected on the swab is performed similarly to that of a surface swab sample.

Prior to the present disclosure, no commercial instruments have used a sampling method where airborne particles are collected directly onto a swab collection substrate.

### SUMMARY OF THE INVENTION

It is an objective of the present invention to provide device and methods that allow for the direct sampling of airborne particles onto a swab collection substrate. Embodiments of the invention are provided to illustrate but not to limit the invention. Embodiments of the present invention can be combined with each other if they are not mutually exclusive.

There have been no commercial applications of sampling airborne particles directly onto a swab prior to this invention. Direct sampling of particles from the air avoids cross contamination from surfaces and sampling biases from preferential settling or filtration according to the particle size. The most direct method of depositing particles from the air onto a solid surface is through inertial impaction. Particles collected by impaction onto a swab from a known volume of air can lead to a quantitative measure of the viral or microbial load, and therefore can inform the investigator about the chemical or biological concentration in the air, including a specific pathogen source strength, exposures and potential risk of infection. As the swab itself can be sterile and not touch any other surfaces, the sample will only have particles that have been deposited from the air. The swab shaft can be held by a mechanical holder so that the extended shaft positions the swab tip directly under the impaction nozzle. In one aspect, the distance from the nozzle exit to the swab surface is about 3 to 5 times the diameter of the nozzle for high efficiency collection. In another aspect, the swab is easily removed from the collection apparatus by touching only the shaft, thus avoiding contamination on the swab tip or posing a safety hazard to the operator (e.g. with exposure to a toxin or infectious pathogen). The swab tip can be removed from the shaft or the shaft cut to remove the swab tip into a vial or vessel for further processing and analysis. A commercial swab enables a simplicity of access to the user as swabs can be purchased from many vendors at a low cost.

Existing sampling devices do not pre-enlarge the particles through condensation growth prior to collection, and therefore collection efficiency drops off considerably with smaller sized particles. Moreover, as the biological particles are collected dry, they will be subject to desiccation, mechanical stress and general degradation of the biological cells and their genetic materials through the process of collecting the sample. This degradation of the sample reduces the recovery and sensitivity of the analysis. As described herein, the present disclosure overcomes the challenges of size-dependent collection efficiency by enlarging all particle to a size that is well above the low-end cut size of the impactor system and by using a swab substrate to improve particle collection for post analysis.

The present disclosure features a device for collecting airborne particles in an air sample. The device may comprise: a) a sample inlet; b) a means for enlarging the particles by condensing supersaturated water vapor onto the particles while the particles are airborne; c) one or more acceleration nozzles coupled to the condensation growth section; and d) a swab collection substrate.

In one embodiment, the swab collection substrate is disposed downstream of the one or more acceleration nozzles. Without wishing to limit the present invention to any theory or mechanism, when an aerosol stream containing the airborne particles is drawn into the means for enlarging the particles through the sample inlet, water vapor is introduced into the aerosol stream creating water vapor supersaturation and condenses onto the airborne particles to form droplets, said droplets having an average diameter larger than the airborne particles in the aerosol stream and the cut size of the impaction system. When the droplets exit the means for enlarging the particles they enter the one or more acceleration nozzles before contacting the swab collection substrate.

In preferred embodiments, substantially all droplets exiting the acceleration nozzles have a sufficient velocity to make contact with the swab collection substrate.

In another embodiment, the swab collection substrate is cantilevered horizontally, vertically, or in any other position that intercepts the aerosol jet stream after exiting from the acceleration nozzles. In one embodiment, the one or more acceleration nozzles is pointing in a downward direction.

In other embodiments, the swab collection substrate comprises a tip and a shaft and is removable from the device. In one embodiment, the swab tip comprises an absorbent material. In a further embodiment, the absorbent material is selected from a group consisting of cotton, polyester, rayon, nylon, polystyrene, synthetic polyurethane foam, or any other material that is absorbent. In another embodiment, the swab tip is round, cylindrical, rectangular, square, paddle shaped, wedge shaped, or any other shape. In yet another embodiment, the swab shaft comprises wood, rolled paper, plastic, or metal. In still another embodiment, the swab tip comprises a well indent. In one embodiment, a second flat substrate is disposed onto the well indent for particle collection.

In some embodiments, the swab collection substrate is pretreated prior to collecting the airborne particles. In other embodiments, the swab collection substrate is pretreated with a buffer, saliva or nasal mucus surrogate, a genomic preservative, or any other matrix comprising salts, proteins, and surfactants to simulate saliva or nasal mucosa. In one embodiment, the swab collection substrate is sterile. In another embodiment, a size of the swab collection substrate is equal to or greater than an inner diameter of the nozzle.

In some embodiments, the airborne particles include aerosolized viruses, bacteria, fungal spores, toxins, metabolites, fragments of biological materials, or a combination thereof.

In one embodiment, the means for enlarging the particles comprises a condensation growth section. In further embodiments, the condensation growth section comprises: a) a conditioner segment; b) an initiator segment; c) a moderator segment; and a wetted wick lining the plurality of walls of the conditioner, initiator, and moderator segments. In one embodiment, the temperature difference between the conditioner and initiator segments are 25° C. or greater. In another embodiment, a temperature of the conditioner segment is about 5 to 10° C. In yet another embodiment, a temperature of the initiator segment is about 35 to 45° C. In still another embodiment, a temperature of the moderator segment is about 8 to 24° C.

The present disclosure also features a method for capturing airborne particles in an air sample. The method may comprise: a) drawing an aerosol sample containing airborne particles into a device, said device comprising a means for enlarging the particles and one or more acceleration nozzles; b) condensing supersaturated water vapor onto the airborne particles while airborne in the means for enlarging the particles, thereby forming droplets having an average diameter larger than the airborne particles; c) expelling the droplets from the means for enlarging the particles from one or more acceleration nozzles; and impacting the droplets onto a swab collection substrate disposed downstream of the one or more acceleration nozzles.

In one embodiment, the method further comprises removing the swab collection substrate from the device and extracting the collected particles from the swab collection substrate for analysis. In some embodiments, the airborne particles are analyzed by ion chromatography, liquid chromatography, polymerase chain reaction (PCR), quantitative PCR (qPCR), reverse transcription PCR (RT-PCR), RT-qPCR, loop mediated isothermal amplification (LAMP), determination of nucleotide sequence of deoxyribonucleic acid, determination of the nucleotides in a strand of ribonucleic acid, immunofluorescence assays, culture assays to determine infectivity, or by other chemical or biological assays.

In some embodiments, the airborne particles include aerosolized viruses, bacteria, fungal spores, toxins, metabolites, fragments of biological materials, or a combination thereof. In another embodiment, an average diameter of the airborne particles is between about 10 to 10,000 nm. In other embodiments, the average diameter of the condensationally-grown droplets is at least one micrometer in diameter.

In another embodiment, the swab collection substrate comprises a tip and a shaft. In some embodiments, the swab tip comprises an absorbent material. In further embodiments, the absorbent material is selected from a group consisting of cotton, polyester, rayon, nylon, polystyrene, synthetic polyurethane foam, or any other material that is absorbent. In yet another embodiment, the swab tip is round, cylindrical, rectangular, square, paddle shaped, wedge shaped, or any other shape. In other embodiments, swab shaft comprises wood, rolled paper, plastic, or metal.

In one embodiment, the swab collection substrate is pretreated prior to collecting the airborne particles. In other embodiments, the swab is pretreated with a buffer, saliva or nasal mucus surrogate, a genomic preservative, or any other matrix comprising salts, proteins, and surfactants to simulate saliva or nasal mucosa. In yet another embodiment, the swab collection substrate is sterile. In other embodiments, a size of the swab collection substrate is equal to or greater than an inner diameter of the nozzle.

In some embodiments, the means for enlarging the particles comprises a condensation growth section. In further embodiments, the condensation growth section comprises: a) a conditioner segment; b) an initiator segment; c) a moderator segment; and a wetted wick lining the plurality of walls of the conditioner, initiator, and moderator segments. In one embodiment, the temperature difference between the conditioner and initiator segments are 25° C. or greater. In another embodiment, a temperature of the conditioner segment is about 5 to 10° C. In yet another embodiment, a temperature of the initiator segment is about 35 to 45° C. In still another embodiment, a temperature of the moderator segment is about 8 to 24° C.

In some embodiments, the swab collection substrate is cantilevered horizontally, vertically, or in any other position that intercepts the aerosol jet stream after exiting from the acceleration nozzles. In another embodiment, the one or more acceleration nozzles is pointing in a downward direction. In one embodiment, the swab tip comprises a well indent. In another embodiment, a second flat substrate is disposed onto the well indent for particle collection.

Any feature or combination of features described herein are included within the scope of the present invention provided that the features included in any such combination are not mutually inconsistent as will be apparent from the context, this specification, and the knowledge of one of ordinary skill in the art. Additional advantages and aspects of the present invention are apparent in the following detailed description and claims.

### BRIEF DESCRIPTION OF THE DRAWINGS

The features and advantages of the present invention will become apparent from a consideration of the following detailed description presented in connection with the accompanying drawings in which:

FIG. 1 is a diagram of a condensation growth tube (CGT) that shows the how airborne particles grow in the different segments of the CGT before being deposited onto a swab.

FIG. 2 shows a flow chart of the process for collecting airborne particles directly onto a swab.

FIG. 3A shows a schematic diagram of conventional impactor.

FIG. 3B shows a corresponding particle collection efficiency curve (Marple and Olson 2011).

FIG. 4A shows a paddle shaped swab with plastic shaft cantilevered and oriented horizontally. The swab tip is directly under the impaction nozzle at the base of a multi-bore condensation growth tube.

FIG. 4B shows a paddle shaped swab cantilevered and oriented horizontally under the impaction nozzle at the base of the condensation growth tube.

FIG. 4C shows a close up of a paddle shaped swab oriented horizontally with swab tip located directly under the impaction nozzle. The swab surface is 3-5 nozzle diameters away from the nozzle exit.

FIG. 5A shows a vertically oriented foam tip swab with a cylindrical tip shape positioned directly under the impaction nozzle. The foam tip can be designed to be easily separated from shaft for analysis of the sample.

FIG. 5B shows a foam tip swab with a well indent at the impaction surface. A circular punch of any substrate material can be held in the bottom of the well as an alternative collection substrate chosen to match the analysis needs. The droplet fluid can be absorbed in the foam swab tip leaving the particle on the substrate.

### DETAILED DESCRIPTION

As used herein, the term “swab collection substrate” refers to a substrate for collecting particles. The particles are typically airborne before they are collected onto the swab collection substrate and the particles may stay on the swab through physical and/or chemical interactions. The term “swab collection substrate” may be used interchangeably with the term “swab”.

One definition of “swab” by The Free Dictionary by Farlex is a “small piece of absorbent material attached to the end of a stick or wire and used for cleansing a surface, applying medicine, or collecting a sample of a substance”. In the present disclosure “swab” is referred to as a noun for the purpose of collecting particles that were once airborne.

Swabs have a variety of household, industrial, forensic, medical and research purposes. In the medical field, sterile swabs are most used to collect a biological sample, or to apply a treatment or disinfectant to minor cuts and abrasions. Nasal, throat and cheek (buccal) swabs are used to collect tissue or fluid from the nose or oral cavity to examine if there is an active infection (using culturing, or a molecular or antigen test), to test saliva for evidence of illegal drug use, or to look at skin cells and saliva for abnormalities to detect cancer.

Medical-grade absorbent swab material is typically made of cotton, polyester, rayon, nylon, polystyrene or synthetic polyurethane foam. They can be flocked (nylon microfibers), foam (reticulated hydrophilic polyurethane), or an organic material treated with calcium alginate (biodegradable, dissolvable). The shafts are typically made of wood, rolled paper or extruded plastic. The swabs can be sterile or non-sterile.

The absorbent properties of swabs can make it difficult to release the material off the swab for molecular analysis. To solve this problem, dissolvable forensic swabs are made of cellulose acetate fibers which are insoluble in water, ethanol and detergent, but are soluble in laboratory DNA extraction buffers that contain chaotropic agents. This allows for smaller samples to be extracted efficiently. The cellulous acetate material is chemically and microbial resistant.

The present disclosure features a device for collecting airborne particles in an air sample. The device may comprise: a) a sample inlet; b) a means for enlarging the particles by condensing supersaturated water vapor onto the particles while the particles are airborne; c) one or more acceleration nozzles coupled to the condensation growth section; and d) a swab collection substrate.

Without wishing to limit the present invention to any theory or mechanism, when the swab collection substrate is disposed downstream of the one or more acceleration nozzles, and when an aerosol stream containing the airborne particles is drawn into the means for enlarging the particles through the sample inlet, water vapor is introduced into the aerosol stream creating water vapor supersaturation, and the water vapor condenses onto the airborne particles to form droplets. The droplets may have an average diameter larger than the airborne particles in the aerosol stream. When the droplets exit the means for enlarging the particles and enter the one or more acceleration nozzles, the droplets are expelled from the acceleration nozzle before contacting the swab substrate via gentle impaction.

The present disclosure also features a method for capturing airborne particles in an air sample. The method may comprise: a) drawing an aerosol sample containing airborne particles into a device, said device comprising a means for enlarging the particles and one or more acceleration nozzles; b) condensing supersaturated water vapor onto the airborne particles while airborne in the means for enlarging the particles, thereby forming droplets having an average diameter larger than the airborne particles; c) expelling the droplets from the means for enlarging the particles from one or more acceleration nozzles; and impacting the droplets onto a swab collection substrate disposed downstream of the one or more acceleration nozzles.

In some embodiments, the method further comprises removing the swab collection substrate from the device and extracting the collected particles from the swab collection substrate for analysis. Non-limiting examples of analysis include ion chromatography, liquid chromatography, polymerase chain reaction (PCR), quantitative PCR (qPCR), reverse transcription PCR (RT-PCR), RT-qPCR, loop mediated isothermal amplification (LAMP), determination of nucleotide sequence of deoxyribonucleic acid, determination of the nucleotides in a strand of ribonucleic acid, immunofluorescence assays, culture assays to determine infectivity, or any other chemical or biological assays.

Examples of the airborne particles include, but are not limited to, aerosolized viruses, bacteria, fungal spores, toxins, metabolites, fragments of biological materials, or a combination thereof. In one embodiment, an average diameter of the airborne particles is between about 10 to 10,000 nm. In another embodiment, the average diameter of the condensationally-grown droplets is at least one micrometer in diameter

In some embodiments, substantially all droplets exiting the one or more acceleration nozzles have a sufficient velocity to make contact with the swab collection substrate. As a non-limiting example, the velocity for droplets 3 micrometers in diameter may be about 24-30 m/s at a separation distance of 3-5 times the inner nozzle diameter for impaction and collection of greater than 90% of the droplets with particles. In another embodiment, the one or more acceleration nozzles is pointing in a downward direction.

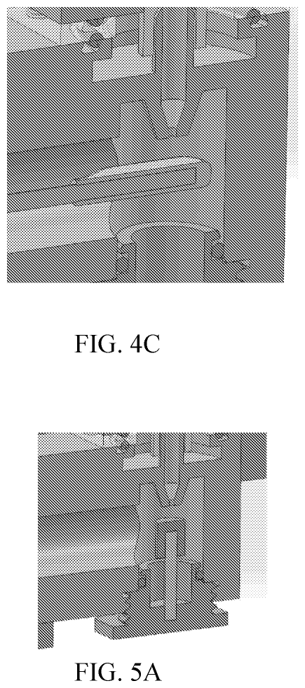

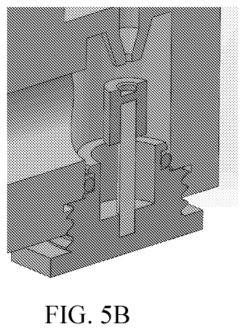

In one embodiment, as shown in FIGS. 4A-4C, the swab collection substrate is cantilevered horizontally under the acceleration nozzle. In another embodiment, as shown in FIGS. 5A-5B, the swab collection substrate is cantilevered vertically under the acceleration nozzle. In yet another embodiment, the swab collection substrate is cantilevered in any position that intercepts the aerosol jet stream after exiting from the acceleration nozzles.

In an embodiment, the swab collection substrate comprises a tip and a shaft and is removable from the device. Without wishing to limit the present invention to any theory or mechanism, the swab collection substrate may be removed from the device to analyze the chemical and/or biological properties of the collected particles. In a further embodiment, the swab tip comprises an absorbent material. Non-limiting examples of absorbent materials include cotton, polyester, rayon, nylon, polystyrene, synthetic polyurethane foam, or any other material that is absorbent and compatible with the desired analysis. Further examples of the absorbent material include, but are not limited to, flocked material such as nylon microfibers, foam such as reticulated hydrophilic polyurethane, or an organic material treated with calcium alginate. In yet another embodiment, the swab tip may be custom shaped foam or another absorbent material.

In some embodiments, the swab tip may be round, cylindrical, rectangular, square, paddle shaped, wedge shaped, or any other shape that provides a collection surface that is at least as large as the size of the acceleration nozzle flow diameter or area. FIG. 5A illustrates a vertically oriented foam swab with a cylindrical tip shape positioned directly under the impaction nozzle. The foam tip can be designed to be easily separated from shaft for analysis of the sample. In other embodiments, the swab tip comprises a well indent. FIG. 5B shows a foam tip with a well indent at the impaction surface. A circular punch of any substrate material can be held in the bottom of the well as an alternative collection substrate chosen to match the analysis needs. The droplet fluid can be absorbed in the foam swab tip leaving the particle on the substrate. In one embodiment, a second flat substrate is disposed onto the well indent for particle collection. In another embodiment, the swab shaft comprises wood, rolled paper, plastic, or metal.

In one embodiment, the swab collection substrate is pretreated prior to collecting the airborne particles. Non-limiting examples of pretreating the swab include pretreating with a buffer, saliva or nasal mucus surrogate, a genomic preservative, or any other matrix comprising salts, proteins, and surfactants to simulate saliva or nasal mucosa. In another embodiment, the swab collection substrate is sterile.

In other embodiments, the means for enlarging the particles comprises a condensation growth section. In further embodiments, the condensation growth section comprises: a) a conditioner segment; b) an initiator segment; c) a moderator segment; and a wetted wick lining the plurality of walls of the conditioner, initiator, and moderator segments. In one embodiment, the temperature difference between the conditioner and initiator segments are 25° C. or greater. In another embodiment, a temperature of the conditioner segment is about 5 to 10° C. In yet another embodiment, a temperature of the initiator segment is about 35 to 45° C. In still another embodiment, a temperature of the moderator segment is about 8 to 24° C.

### EXAMPLE

The following is a non-limiting example of the present invention. It is to be understood that said example is not intended to limit the present invention in any way. Equivalents or substitutes are within the scope of the present invention.

### Pretreatment of the Collection Substrate for Viability Assays

Similar to the pretreatment with genomic preservative described above, the absorbent swab tip can be pretreated with a buffer, saliva, artificial saliva or nasal mucus, saliva surrogate, or other suitable liquid matrix to help maintain microbial and virus viability.

### Implementation:

### Moderated, Laminar-Flow Water Condensation Growth Tube (CGT) and Biological Particle Collection

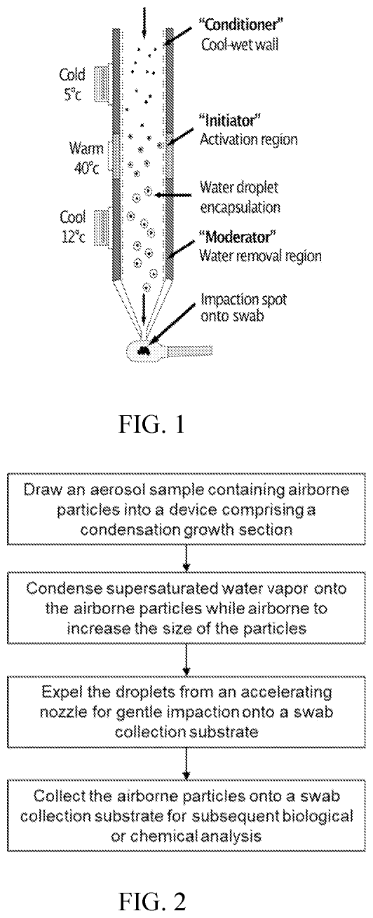

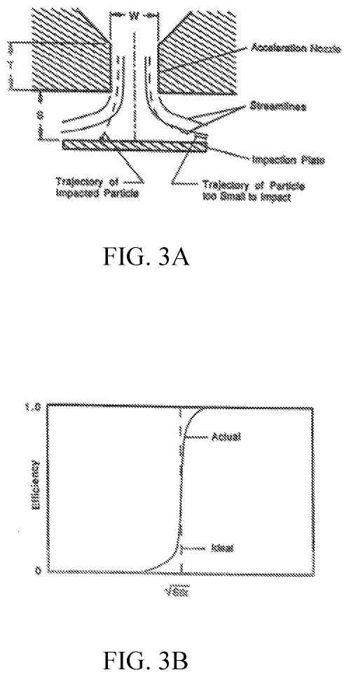

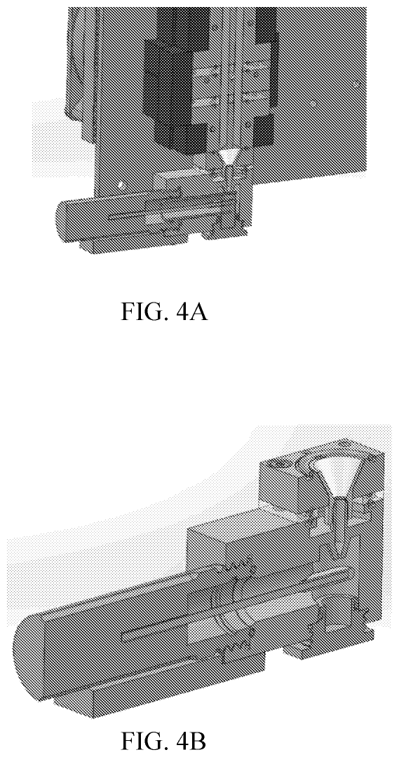

The existing commercial Liquid Spot Sampler™ aerosol particle collector from Aerosol Devices Inc. is uniquely capable of collecting delicate biological particles extremely efficiently while maintaining their viability. The core sampling technology is a three-stage laminar-flow, water-based condensational growth tube (Eiguren Fernandez et al 2014a; Hering and Stolzenburg 2005; Pan et al. 2016) (FIG. 1) that collects and concentrates virtually all bioaerosol particles from <10-10,000 nm onto a solid surface or into a small volume of liquid (˜0.5 mL), making it vastly more effective than other samplers at capturing the bare viruses (20-300 nm), bacteria and fungal spores (>300-10000 nm) as well as viruses and bacteria encased in droplet secretions (0.2 to 10 μm).

The condensation growth tube's (CGT's) moderate temperatures and humidity mimic the environment in the human lung. Particles enter the CGT and move through the cold (5° C.) conditioner, which establishes a controlled water vapor saturated sample stream independent of ambient conditions. Supersaturation occurs downstream of the conditioner in the initiator as a result of the difference between the diffusivity of water vapor and thermal energy. The warm (35-45° C.) walls of the initiator heat the sample air and increase the partial pressure of water vapor, but since water vapor diffuses more rapidly in air than thermal energy, the water vapor diffuses into the flow faster than the flow warms. Thermodynamic equilibrium then drives the supersaturated air to condense water vapor on the seed particles. The high supersaturation ratio in the initiator (˜140%) activates condensational growth of particles as small as 5-10 nm in diameter. Once activated, the particles grow through condensation as they pass through the final moderator section (8-24° C.) to form ˜3 μm droplets that are readily collected with gentle, low-velocity impingement into a liquid (water, buffer, or nutrient broth) or onto a solid surface. The jet velocity for 3 micrometer droplets is approximately 30 m/s at a separation distance of 3-5 times the inner nozzle diameter for impaction or impingement collection efficiency of greater than 90%. The warm liquid medium and gentle impingement into liquid prevents desiccation or mechanical stresses, protecting the microorganisms and maintaining infectivity/viability. The very low liquid output from the CGT ensures a highly concentrated bioaerosol sample, reducing the sample time required for good detection sensitivity.

Some of the advantages of CGT bioaerosol particle collection include: 1) Direct, gentle collection into liquid or onto a solid substrate improves virus and bacteria recovery using molecular analysis and culturing; 2) Uniform high collection efficiency for particle sizes from bare viruses to inhalable droplets up to 10 micrometers diameter; and 3) High sample concentration reduces sampling time and/or increases detection sensitivity.

The device of the present invention employs sampling directly onto swabs, as well as onto other solid substrates and into liquid. The swab can be oriented vertically (FIGS. 4A-4C), horizontally (FIGS. 5A-5B), or any orientation that is directly downstream of the impaction nozzle, and approximately 3-5 nozzle diameters distance between the nozzle exit and the swab surface.

Although there has been shown and described the preferred embodiment of the present invention, it will be readily apparent to those skilled in the art that modifications may be made thereto which do not exceed the scope of the appended claims. Therefore, the scope of the invention is only to be limited by the following claims. In some embodiments, the figures presented in this patent application are drawn to scale, including the angles, ratios of dimensions, etc. In some embodiments, the figures are representative only and the claims are not limited by the dimensions of the figures. In some embodiments, descriptions of the inventions described herein using the phrase “comprising” includes embodiments that could be described as “consisting essentially of” or “consisting of”, and as such the written description requirement for claiming one or more embodiments of the present invention using the phrase “consisting essentially of” or “consisting of” is met.

### REFERENCES

All literatures and patents or patent applications cited here or throughout the disclosure are hereby incorporated by reference in this disclosure.
Ahn, K, H. 2010. U.S. Pat. No. 7,724,368 B2, Condensation Particle Counter. Aitken, John, 1888. On the number of dust particles in the atmosphere, Proc. Royal Soc Edinburgh.: 1-19.
Cheng, Y. S. 2011. Condensation Particle Counters. Aerosol Measurement, edited by P. Kulkarni, P. Baron and K. Willeke, pages 381-392.
Eiguren Fernandez, A., G. S. Lewis & S. V. Hering. 2014a. Design and Laboratory Evaluation of a Sequential Spot Sampler for Time-Resolved Measurement of Airborne Particle Composition. Aerosol Science and Technology, 48:6, 655-663, DOI: 10.1080/02786826.2014.911409.

Eiguren-Fernandez, A., Lewis, G. S., Spielman, S. R., Hering, S. V. 2014b. Time-resolved Characterization of Particle Associated Polycyclic Aromatic Hydrocarbons using a newly-developed Sequential Spot Sampler with Automated Extraction and Analysis. Atmospheric Environment, Vol 96, pp 125-134, DOI: 10.1016/j.atmosenv.2014.07.031.
Hecobian, A., Evanoski-Cole, A., Eiguren-Fernandez, A., Sullivan, A. P., Lewis, G. S., Hering, S. V and Collett Jr., J. L. 2015. Evaluation of a Sequential Spot Sampler (S3) for time-resolved measurement of PM2.5 sulfate and nitrate through lab and field measurements, Atmos. Meas. Tech. Discuss., 8, 10611-10633, DOI:10.5194/amtd-8-10611-20
Hering, S. V. and M. R. Stolzenburg. 2005. A Method for Particle Size Amplification by Water Condensation in a Laminar, Thermally Diffusive Flow. Aerosol Science and Technology, 39:428-436, DOI: 10.1080/027868290953416
Hering, S. V., Spielman, S. R., and Lewis, G. L. 2014 Moderated, water-based condensational growth of particles in a laminar flow. Aerosol Science and Technology 48:401-40, DOI:10.1080/02786826.2014.881460.
Hernandez, M. and P. Keady. 2019. WO 2019/133718A1 International Patent Application, Hi-Fidelity Bioaerosol Condensation Capture Directly Into Genomic Preservative.
Jensen, P. A. and M. P. Schafer. 1998. Sampling and Characterization of Bioaerosols, NIOSH Manual of Analytical Methods. [PFP #492479137] (cdc.gov), https://www.cdc.gov/niosh/docs/2003-154/pdfs.chapter-j/pdf
Jansson, L., Y. Akel, R. Eriksson, M. Lavander, J. Hedman. 2020. Impact of swab material on microbial surface sampling, Journal of Microbiological Methods, Volume 176, 106006, ISSN 0167-7012, https://doi.org/10.1016/j.mimet.2020.106006.
Kim H R, An S, Hwang J, Park J H, Byeon J H. In situ lysis droplet supply to efficiently extract ATP from dust particles for near-real-time bioaerosol monitoring. J Hazard Mater. 2019 May 5; 369:684-690. doi: 10.1016/j.jhazmat.2019.02.088. Epub 2019 Feb. 25. PMID: 30826561.
Liao L, Byeon J H, Park J H. Development of a size-selective sampler combined with an adenosine triphosphate bioluminescence assay for the rapid measurement of bioaerosols. Environ Res. 2021 March; 194:110615. doi:10.1016/j.envres.2020.110615. Epub 2020 Dec. 10. PMID: 33309960.
Lewis, G. S., and S. V. Hering. 2013 Minimizing Concentration Effects in Water-Based, Laminar-Flow Condensation Particle Counters, Aerosol Science and Technology, 47:6, 645-654, DOI: 10.1080/02786826.2013.779629
Marple, V. A. 1970. A Fundamental Study of Inertial Impactors. Doctoral Thesis, University of Minnesota, Minneapolis, MN.
Marple, V. A. and B. Y. H. Liu. 1974. Characteristics of laminar jet impactors. Environ. Sci. Technol. 14:976-985.
Marple, V. A. 2004. History of impactors—The First 100 years. Aerosol Sci. Technol. 38:247-292.
Marple, V. A. and B. A. Olson. 2011. Sampling and Measurement Using Inertial Gravitational, Centrifugal, and Thermal Techniques, Aerosol Measurement, edited by P. Kulkarni, P. Baron and K. Willeke, pages 129-151.
McMurry, Peter. 2000. The history of condensation nucleus counters. Aerosol Sci. Technol. 33:297-322.
MetsSub, citizen science project Weill Cornell Medicine|MetaSUB: Discover and Map the Genomes Around Us, https://crowdfunding.cornell.edu/project/3636.
Nieto-Caballero, M., N. Savage, P. Keady, M. Hernandez. 2018. High fidelity recovery of airborne microbial genetic materials by direct condensation capture into genomic preservatives, Journal of Microbiological Methods, 157, 1-3, https://doi.org/10.1016/j.mimet.2018.12.010.
Pan, M., A. Eiguren-Fernandez, H. Hsieh, N. Afshar-Mohajer, S. V. Hering, J. Lednicky, Z. Hugh Fan and C.-Y. Wu. 2016. Efficient collection of viable virus aerosol through laminar-flow, water-based condensational particle growth, Journal of Applied Microbiology, Volume 120, Issue 3, March 2016, Pages 805-815, DOI: 10.1111/jam.13051.
Romay, F. J., A. Collins, W. D. Dick, L. Li, C. W. Fandrey and B. Y. H. Liu. 2016. Water-based single-flow mixing condensation particle counter, Aerosol Science and Technology, 50:12, 1320-1326, DOI: 10.1080/02786826.2016.1222510.
Van Schooneveld, G. S., P. Keady, and D. Blackford (2018) The Use of Focused Aerosol Deposition (FAD) to capture, identify and quantify killer defect particles in UPW. Ultrapure Water Micro 2018, Austin, Texas, May 31.
Weber, R. J., D. Orsini, Y. Daun, Y.-N. Lee, P. J. Klotz & F. Brechtel. 2001. A Particle-into-Liquid Collector for Rapid Measurement of Aerosol Bulk Chemical Composition, Aerosol Science & Technology, 35:3, 718-727, DOI: 10.1080/02786820152546761
WHO. 2020. Surface sampling of coronavirus disease (COVID-19): A practical “how to” protocol for health care and public health professionals, WHO/2019-nCoV/Environment_protocol/2020.1
Yamayoshi, T., H. Doi, N. Tatsumi. 1984. Surface sampling using a single swab method, Journal of Hospital Infection, Volume 5, Issue 4, Pages 386-390, ISSN 0195-6701, https://doi.org/10.1016/0195-6701(84)90006-9.
Zheng, L., P. Kulkarni, M. Eileen Birch, K. Ashley, and S. Wei. 2018. Analysis of Crystalline Silica Aerosol Using Portable Raman Spectrometry: Feasibility of Near Real-Time Measurement, Analytical Chemistry 90 (10), 6229-6239 DOI: 10.1021/acs.analchem.8b00830.

We claim:

1. A device for collecting airborne particles in an air sample, comprising:
a sample inlet
a means for enlarging the particles by condensing supersaturated water vapor onto the particles while the particles are airborne;
one or more acceleration nozzles coupled to the means for enlarging the particles; and
a swab collection substrate;
wherein the swab collection substrate is disposed downstream of the one or more acceleration nozzles, and wherein when an aerosol stream containing the airborne particles is drawn into the means for enlarging the particles through the sample inlet, water vapor is introduced into the aerosol stream creating water vapor supersaturation and condenses onto the airborne particles to form droplets, said droplets having an average diameter larger than the airborne particles in the aerosol stream, wherein the droplets exit the means for enlarging the particles and enter the one or more acceleration nozzles before contacting the swab collection substrate.

2. The device of claim 1, wherein substantially all droplets exiting the acceleration nozzles have a sufficient velocity to make contact with the swab collection substrate.

3. The device of claim 1, wherein the swab collection substrate is cantilevered horizontally, vertically, or in any other position that intercepts the aerosol jet stream after exiting from the acceleration nozzles.

4. The device of claim 1, wherein the one or more acceleration nozzles is pointing in a downward direction.

5. The device of claim 1, wherein the swab collection substrate comprises a tip and a shaft, and is removable from the device.

6. The device of claim 5, wherein the swab tip comprises an absorbent material.

7. The device of claim 6, wherein the absorbent material is selected from a group consisting of cotton, polyester, rayon, nylon, polystyrene, synthetic polyurethane foam, or any other material that is absorbent.

8. The device of claim 5, wherein the swab tip is round, cylindrical, rectangular, square, paddle shaped, wedge shaped, or any other shape.

9. The device of claim 5, wherein the swab shaft comprises wood, rolled paper, plastic, or metal.

10. The device of claim 5, wherein the swab tip comprises a well indent.

11. The device of claim 10, wherein a second flat substrate is disposed onto the well indent for particle collection.

12. The device of claim 1, wherein the swab collection substrate is pretreated prior to collecting the airborne particles.

13. The device of claim 12, wherein the swab collection substrate is pretreated with a buffer, saliva or nasal mucus surrogate, a genomic preservative, or any other matrix comprising salts, proteins, and surfactants to simulate saliva or nasal mucosa.

14. The device of claim 1, wherein the swab collection substrate is sterile.

15. The device of claim 1, wherein a size of the swab collection substrate is equal to or greater than an inner diameter of the nozzle.

16. The device of claim 1, wherein the airborne particles include aerosolized viruses, bacteria, fungal spores, toxins, metabolites, fragments of biological materials, or a combination thereof.

17. The device of claim 1, wherein the means for enlarging the particles comprises:
a conditioner segment;
an initiator segment;
a moderator segment; and
a wetted wick lining the plurality of walls of the conditioner, initiator, and moderator segments.

18. The device of claim 17, wherein the temperature difference between the conditioner and initiator segments are 25° C. or greater.

19. The device of claim 17, wherein a temperature of the conditioner segment is about 5 to 10° C.

20. The device of claim 17, wherein a temperature of the initiator segment is about 35 to 45° C.

21. The device of claim 17, wherein a temperature of the moderator segment is about 8 to 24° C.

22. A method for capturing airborne particle samples in an air sample, comprising:
a. drawing an aerosol sample containing airborne particles into a device, said device comprising a means for enlarging the particles and one or more acceleration nozzles;
b. condensing supersaturated water vapor onto the airborne particles while airborne in the means for enlarging the particles, thereby forming droplets having an average diameter larger than the airborne particles,
c. expelling the droplets from the means for enlarging the particles from one or more acceleration nozzles; and
d. impacting the droplets onto a swab collection substrate disposed downstream of the one or more acceleration nozzles.

23. The method of claim 22, further comprising removing the swab collection substrate from the device and extracting the collected particles from the swab collection substrate for analysis.

24. The method of claim 23, wherein the airborne particles are analyzed by ion chromatography, liquid chromatography, polymerase chain reaction (PCR), quantitative PCR (qPCR), reverse transcription PCR (RT-PCR), RT-qPCR, loop mediated isothermal amplification (LAMP), determination of nucleotide sequence of deoxyribonucleic acid, determination of the nucleotides in a strand of ribonucleic acid, immunofluorescence assays, culture assays to determine infectivity, or by other chemical or biological assays.

25. The method of claim 22, wherein the airborne particles include aerosolized viruses, bacteria, fungal spores, toxins, metabolites, fragments of biological materials, or a combination thereof.

26. The method of claim 22, wherein the swab collection substrate comprises a tip and a shaft.

27. The method of claim 26, wherein the swab tip comprises an absorbent material.

28. The method of claim 27, wherein the absorbent material is selected from a group consisting of cotton, polyester, rayon, nylon, polystyrene, synthetic polyurethane foam, or any other material that is absorbent.

29. The method of claim 26, wherein the swab tip is round, cylindrical, rectangular, square, paddle shaped, wedge shaped, or any other shape.

30. The method of claim 26, wherein the swab shaft comprises wood, rolled paper, plastic, or metal.

31. The method of claim 22, wherein the swab collection substrate is pretreated prior to collecting the airborne particles.

32. The method of claim 31, wherein the swab is pretreated with a buffer, saliva or nasal mucus surrogate, a genomic preservative, or any other matrix comprising salts, proteins, and surfactants to simulate saliva or nasal mucosa.

33. The method of claim 22, wherein the swab collection substrate is sterile.

34. The method of claim 22, wherein a size of the swab collection substrate is equal to or greater than an inner diameter of the nozzle.

35. The method of claim 22, wherein an average diameter of the airborne particles is between about 10 to 10,000 nm.

36. The method of claim 22, wherein the average diameter of the condensationally-grown droplets is at least one micrometer in diameter.

37. The method of claim 22, wherein the condensation growth section comprises:
a conditioner segment;
an initiator segment;
a moderator segment; and
a wetted wick lining the plurality of walls of the conditioner, initiator, and moderator segments.

38. The method of claim 37, wherein the temperature difference between the conditioner and initiator segments are 25° C. or greater.

39. The method of claim 37, wherein a temperature of the conditioner segment is about 5 to 10° C.

40. The method of claim 37, wherein a temperature of the initiator segment is about 35 to 45° C.

41. The method of claim 37, wherein a temperature of the moderator segment is about 8 to 24° C.

42. The method of claim 22, wherein the swab collection substrate is cantilevered horizontally, vertically, or in any other position that intercepts the aerosol jet stream after exiting from the acceleration nozzles.

43. The method of claim 22, wherein the one or more acceleration nozzles is pointing in a downward direction.

44. The method of claim 26, wherein the swab tip comprises a well indent.

45. The method of claim 44, wherein a second flat substrate is disposed onto the well indent for particle collection.
